(19) United States
(12) Patent Application Publication
(10) Pub. No.: US 2007/0299089 A1
(43) Pub. Date: Dec. 27, 2007

(54) Use of A2A Adenosine Receptor Agonists in the Treatment of Ischemia

(75) Inventors: Luiz Belardinelli, Palo Alto, CA (US); Arvinder Dhalla, Mountain View, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CV THERAPEUTICS, INC.

(21) Appl. No.: 11/766,964

(22) Filed: Jun. 22, 2007

Related U.S. Application Data
(60) Provisional application No. 60/815,755, filed on Jun. 22, 2006.

(51) Int. Cl. A61K 31/52

(52) U.S. Cl. 514/263.2

(57) ABSTRACT

Methods for treating ischemia in mammals with one or more compounds that are agonists of mammalian adenosine A_{2A} receptors.

### FIELD OF THE INVENTION

This invention relates to methods for reducing ischemia in mammals by administering an adenosine A_{2A} receptor agonist such as regadenoson to a mammal wherein the adenosine A_{2A} receptor agonist is administered in an amount sufficient to attenuate the ischemia.

### BACKGROUND

Coronary microcirculatory vasoconstriction has been documented during ischemia in humans with severe coronary stenosis. The vasoconstriction contributes to the worsening of ischemia. Pharmacological agents that increase cardiac blood flow (CBF) for a short period of time would be of great benefit in treating ischemia.

Potent and selective agonists for the A_{2A} adenosine receptor are known in the art and are described as being useful in pharmaceutical compositions for many reasons including for use in conjunction with coronary imaging agents.

One especially potent and useful adenosine A_{2A} receptor agonist is regadenoson ((1-{9-[(2R,3R,4S,5R)-3,4-dihydroxy-5-(hydroxymethyl)oxolan-2-yl]-6-aminopurin-2-yl}pyrazol-4-yl)-N-methylcarboxamide). Regadenoson is selective for the adenosine A_{2A} receptor, has a short duration of action and does not appear to require administration as a continuous infusion. Regadenoson and related A_{2A} receptor agonist compounds as well as methods for their manufacture and use in cardiac perfusion imagining are disclosed in U.S. Pat. Nos. 6,403,567, 6,642,210, 6,214,807, and 6,770,634, 7,109,180, 7,144,872, and 7,183,264, the entirety of each specification of which are incorporated herein by reference. Other Although regadenoson is a known compound, much remains unknown about its pharmacokinetic profile and range of potential therapeutic uses.

### SUMMARY OF THE INVENTION

One aspect of this invention is a method of treating ischemia in a mammal comprising administering to the mammal a therapeutically effective amount of at least one compound that is an A_{2A} receptor agonist.

In another aspect, this invention is a method for reducing coronary ischemia in a human patient comprising administering at least one dose of a pharmaceutical composition comprising (1-{9-[(2R,3R,4S,5R)-3,4-dihydroxy-5-(hydroxymethyl)oxolan-2-yl]-6-aminopurin-2-yl}pyrazol-4-yl)-N-methylcarboxamide and at least one pharmaceutical excipient to the human patient in an amount sufficient to reduce the human patient's coronary perfusion pressure wherein the coronary ischemia is accompanied by coronary microcirculatory vasoconstriction.

The methods of this invention may be accomplished, in one aspect, using a pharmaceutical composition comprising the A_{2A} receptor agonist compound and one or more pharmaceutical excipients.

### DESCRIPTION OF A PREFERRED EMBODIMENT

This invention is directed to methods for treating ischemia in mammals with one or more compositions that are A_{2A} receptor agonists. As used herein the term A_{2A} receptor agonists refers to compounds that are agonists of mammalian adenosine A_{2A} receptors.

Ischemia is a restriction in blood supply resulting in a shortage of blood supply to one or more organs of a mammal. The shortage of blood supply results in a lack of oxygenation to the organ(s) and causes them to become hypoxic, or if no oxygen at all is being supplied, anoxic. Prolonged ischemia can result in organ damage and/or death.

The methods of this invention are useful for treating ischemia occurring anywhere in a mammal body. In particular the methods of this invention are useful for treating ischemia affecting one or more of the heart, brain and/or kidneys and they are especially useful for treating coronary ischemia—ischemia affecting the heart muscle. Coronary ischemia is often accompanied by coronary microcirculatory vasoconstriction—a constriction of the blood vessels comprising the microcirculatory system of the heart. Microcirculatory vasoconstriction causes a reduction in blood flow to at least a portion of the heart. One way to attenuate or reverse ischemia is to increase blood flow to the affected area or organ. With coronary ischemia a preferred method is to increase blood flow to the affected area without increasing CPP.

The methods of this invention employ A_{2A} receptor agonists to increase blood flow in part by dilating blood vessels in the affected area or organ, and, in the case of coronary microcirculatory vasoconstriction, by coronary vasodilation. It is preferred, but not required, that when treating coronary ischemia by the methods of this invention that the A_{2A} receptor agonist be selected from compounds that promote coronary vasodilation but do not increase peripheral blood flow in a statistically significantly way.

The methods of this invention employ pharmaceutical compositions that include at least one compound that is an A_{2A} adenosine receptor agonist that promotes coronary vasodilatation in the ischemic area or organ. Any A_{2A} receptor agonist may be useful in the methods of this invention. Two especially useful A_{2A} receptor agonists are designated herein as CVT-3146 and CVT-3033. Both compounds have a rapid onset and a short duration when administered. Moreover, the preferred compounds reduce coronary vascular resistance but do not significantly increase peripheral blood flow.

CVT-3146 is known alternatively as regadenoson. Regadenoson is also known as (1-{9-[(4S,2R,3R,5R)-3,4-dihydroxy-5-(hydroxymethyl)oxolan-2-yl]-6-aminopurin-2-yl}pyrazol-4-yl)-N-methylcarboxamide and has the formula: 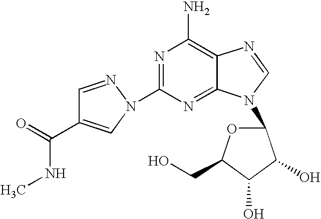
Methods for synthesizing regadenoson and related compounds are set forth in U.S. Pat. No. 6,403,567, the specification of which is incorporated herein by reference in its entirety.

CVT-3033 is also known as also known as (4S,2R,3R,5R)-2-[6-amino-2-(1-pentylpyrazol-4-yl)purin-9-yl]-5-(hydroxymethyl)oxolane-3,4-diol and having the formula: 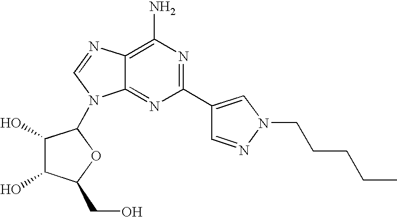
Methods for synthesizing CVT-3033 and related A_{2A} receptor antagonist compounds are set forth in U.S. Pat. No. 6,214,807, the specification of which is incorporated herein by reference in its entirety.

The adenosine A_{2A} receptor agonist compounds of this invention are administered in a therapeutically effective amount to a mammal. The term “therapeutically effective amount” refers to that amount of an A_{2A} receptor agonist, such as a compound of Formula I that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

In one embodiment, the therapeutically effective amount should be sufficient to increase vasodilation in the ischemic area. With coronary ischemia, a therapeutically effective amount should improve vasodilation and blood flow in the affected area—preferably without an increase in coronary perfusion pressure. Alternatively, the therapeutically effective amount should decrease coronary perfusion pressure by at least 10% and more preferably should bring coronary perfusion pressure back to about the baseline pressure—i.e. the coronary perfusion pressure preceding the ischemic event.

The term “treatment” or “treating” means any treatment of a disease in a mammal, including:

(i) preventing the disease, that is, causing the clinical symptoms of the disease not to develop;

(ii) inhibiting the disease, that is, arresting the development of clinical symptoms; and/or

(iii) relieving the disease, that is, causing the regression of clinical symptoms.

Pharmaceutical Compositions

Therapeutically effective amounts of A_{2A} receptor agonist compounds are preferably administered in the methods of this invention in the form of pharmaceutical compositions. The methods of this invention therefore provide pharmaceutical compositions that contain, as the active ingredient, one or more A_{2A} receptor agonists, and one or more pharmaceutically acceptable excipients, carriers, including inert solid diluents and fillers, diluents, including sterile aqueous solution and various organic solvents, permeation enhancers, solubilizers and adjuvants. The compounds of Formula I may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17.sup.th Ed. (1985) and “Modern Pharmaceutics”, Marcel Dekker, Inc. 3.sup.rd Ed. (G. S. Banker & C. T. Rhodes, Eds.).

Administration

The A_{2A} receptor agonist compounds and pharmaceutical compositions containing A_{2A} receptor agonists may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof, cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating one or more A_{2A} receptor agonist compound(s) in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration according to the methods of this invention. Administration may be via capsule or enteric coated tablets, or the like. In making the pharmaceutical compositions that include at least one compound of Formula I, the active ingredient is usually diluted by an excipient and/or enclosed within such a carrier that can be in the form of a capsule, sachet, paper, or other container. When the excipient serves as a diluent, it can be a solid, semi-solid, or liquid material (as above), which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, and ampoule). The pharmaceutical compounds used in the methods of this invention are effective over a wide dosage range and are generally administered in a pharmaceutically effective amount.

When administered in a very small quantity in a single bolus intravenous (i.v.) injection, the A_{2A} receptor agonists can be administered in amounts as little as 10 μg and as high as 600 μg or more and still be effective with few if any side-effects. An optimal dose may include as little as 10 μg and as much as about 1000 μg or more of an A_{2A} receptor agonist.

Alternatively, the pharmaceutical composition including an effective amount of an A_{2A} receptor agonist may be administered by continuous i.v. infusion. With a continuous i.v. infusion, an effective dose will range from about 1 to about 500 μg/min of at least one A_{2A} receptor agonist.

In one aspect of this invention, mammalian ischemia is treated with a pharmaceutical composition including CVT-3146—regadenoson. Regadenoson has solubility in water of about 50 micrograms/mL. Therefore, regadenoson can be dissolved and administered in water so long as the desired weight amount of regadenoson can be administered in an acceptable volume. For example, a dose of about 400 micrograms can be administered in 8 mL of water. If this volume is too great for administration purposes, or if the pharmaceutical composition will be stored at other than room temperature (RT), then additional ingredients can be added to the composition to increase the solubility of regadenoson in the composition and/or to provide the resulting pharmaceutical composition with other improved properties such as improved stability and storage properties.

Liquid pharmaceutical compositions of this invention that include regadenoson may include up to about 1 milligram/mL of regadenoson. It is preferred that pharmaceutical compositions including regadenoson include from about 50 to about 250 micrograms/mL, and more preferably from about 50 to 150 micrograms/mL of regadenoson.

In order to improve solubility and storage properties, regadenoson can be administered in a pharmaceutical composition including a methylboronic acid (MBA) co-solvent. The methylboronic acid is added to the pharmaceutical composition to improve agonist solubility and shelf life. MBA increases the pH of the resulting composition. The solubility of regadenoson in a pharmaceutical composition including MBA tends to decrease as the composition pH drops towards neutral. Therefore, with regadenoson, an optimal MBA-containing composition pH is from about 8.5 to 10 with a pH of about 9.1 to about 9.4 being preferred and a pH of about 9.3 being most preferred. This corresponds to a composition including from about 50 to about 250 mg/mL of MBA. As an alternative to MBA, regadenoson can be combined with a borate buffer solution. Typically, a borate buffer solution will be comprised of an aqueous solution of sodium borate that is adjusted to the desired pH such as a pH of 9.3 using an acid or a base.

MBA containing pharmaceutical compositions can suffer from storage problems. Namely, MBA can cause delamination when packaged in certain type I glass vessels. This problem can be overcome by storing the MBA containing pharmaceutical compositions in plastic vessels or in more resistant type I glass vessels.

If regadenoson containing pharmaceutical compositions having a pH closer to neutral are desired, then an alternative is to combine regadenoson with a propylene glycol (PG) co-solvent. The amount of PG used in the composition may range from about 5% to up to 25% by volume with a range of about 8% to about 20% by volume being more preferred when using regadenoson. An alternative to PG is polyethylene glycol—PEG. A preferred PEG will have an average molecular weight of from about 200 to 400.

Preferably, the regadenoson composition including PG or PEG will have a pH of from about 6 to about 8 with a pH of about 7 being preferred. Any physiologically acceptable buffer capable of adjusting the composition pH to the desired value can be used. Examples of such buffer include, but are not limited to, dibasic sodium phosphate, dibasic sodium phosphate dehydrate, and monobasic sodium phosphate monohydrate. Additional optional ingredients such as EDTA and dimethylacetamide could be employed in the composition as well.

For oral administration, each dosage unit may contain from 10 mg to 2 g of an A_{2A} receptor agonist compound, more preferably from 10 to 700 mg, and for parenteral administration, preferably from 10 to 700 mg, more preferably about 50-200 mg. It will be understood, however, that the amount of the selected compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid pharmaceutical compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills, and capsules.

Tablets or pills used in the methods of the of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Therapeutically effective compositions administered by inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The A_{2A} agonists of this invention are preferably administered in a single dose. The term “single dose” refers generally to a single quickly administered dose of a therapeutic amount of at least one A_{2A} receptor agonist. The term “single dose” does not encompass a dose or doses administered over an extended period of time by, for example continuous i.v. infusion.

Pharmaceutical compounds used in the methods of this invention may be formulated as solutions or lyophilized powders for parenteral administration. Powders may be reconstituted by addition of a suitable diluent or other pharmaceutically acceptable carrier prior to use. As used herein, “pharmaceutically acceptable carrier” includes all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. If used in liquid form the compounds of this invention are preferably incorporated into a buffered, isotonic, aqueous solution. Examples of suitable diluents are normal isotonic saline solution, standard 5% dextrose in water and buffered sodium or ammonium acetate solution. Such liquid formulations are suitable for parenteral administration, but may also be used for oral administration. It may be desirable to add excipients such as polyvinylpyrrolidinone, gelatin, hydroxy cellulose, acacia, polyethylene glycol, mannitol, sodium chloride, sodium citrate or any other excipient known to one of skill in the art to pharmaceutical compositions including compounds of this invention.

Pharmaceutical compositions used in the methods of this invention may be prepared and then administered, with or without intervening storage. Various properties considered when formulating pharmaceutical compositions of this invention include, but are not limited to product shelf life, A_{2A} receptor agonist solubility, composition pH, vein irritation, hemolysis, storage conditions (e.g., whether the pharmaceutical composition will be stored at room temperature or some other temperature) and the ability to withstand sterilization procedures.

The following example is included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

The objective of this example was to determine whether the selective and stable A_{2A} agonist, regadenoson, can prevent or modify coronary microcirculation vasoconstriction during ischemia. To this purpose, in 60 mouse isolated hearts perfused in Langendorff configuration coronary vascular resistance (CVR) was continuously monitored for 70 min during various interventions using two different protocols. In hearts (n=9) perfused at a constant coronary perfusion pressure (CPP) of 65 mmHg CVR remained stable throughout the 70 min of the experiment.

In Protocol 1, CPP was lowered to 30 mmHg at 20 min, maintained for the following 20 min and restored to 65 mmHg for the remaining 30 min. In untreated hearts (n=12), lowering CPP for 20 min increased CVR up to a maximum of 191%±9 above baseline (P<0.001). Regadenoson markedly attenuated this increase in CVR at the concentration of 3 nM (n=6, maximum of 131%±15 above baseline) and abolished the increase in CVR at 10 and 30 nM (n=6; p<0.001 for each concentration). In untreated hearts, restoring CPP to 65 mmHg caused a sudden but incomplete vasodilation that resulted in a lower CVR that was 153%±10 above baseline. This response was significantly reduced by 3 nM regadenoson and abolished by 10 and 30 nM (p<0.01).

In Protocol 2, CPP was lowered to 30 mmHg at 20 min and maintained for the remaining 50 min. In untreated hearts (n=9), the sustained lowering of CPP was accompanied by a progressive rise in CVR up to the end of the experiment when it reached a maximum of 240%±21 above baseline. Addition of regadenoson (10 nM) to the perfusate either at 10 (n=6) or 30 (n=6) min after lowering CPP caused a rapid and progressive vasodilation thereby bringing CVR to baseline values within 10 min. In summary, the isolated heart reacts to lowering of CPP with a progressive and severe vasoconstriction that can be prevented by pre-treatment with an A_{2A} agonist. Further, activation of A_{2A} adenosine receptors during the vasoconstriction can blunt and reverse this microvascular reaction and hence improve myocardial perfusion and reduce the severity of ischemia.

What is claimed is:

1. A method of treating ischemia in a mammal comprising administering to the mammal a therapeutically effective amount of at least one compound that is an A_{2A} receptor agonist.

2. The method of claim 1 wherein the ischemia treated is ischemia affecting one or more organs selected from the group consisting of the heart, the brain and the kidneys.

3. The method of claim 1 wherein the ischemia treated is coronary ischemia.

4. The method of claim 3 wherein the coronary ischemia is accompanied by microcirculatory vasoconstriction.

5. The method of claim 1 wherein the mammal is a human.

6. The method of claim 1 wherein the A_{2A} receptor agonist is (1-{9-[(4S,2R,3R,5R)-3,4-dihydroxy-5-(hydroxymethyl)oxolan-2-yl]-6-aminopurin-2-yl}pyrazol-4-yl)-N-methylcarboxamide, (4S,2R,3R,5R)-2-[6-amino-2-(1-pentylpyrazol-4-yl)purin-9-yl]-5-(hydroxymethyl)oxolane-3,4-diol, or a mixture thereof.

7. The method of claim 1 wherein the A_{2A} receptor agonist is (1-{9-[(4S,2R,3R,5R)-3,4-dihydroxy-5-(hydroxymethyl)oxolan-2-yl]-6-aminopurin-2-yl}pyrazol-4-yl)-N-methylcarboxamide.

8. A method for reducing coronary ischemia in a human patient comprising administering at least one dose of a pharmaceutical composition comprising (1-{9-[(2R,3R,4S,5R)-3,4-dihydroxy-5-(hydroxymethyl)oxolan-2-yl]-6-aminopurin-2-yl}pyrazol-4-yl)-N-methylcarboxamide and at least one pharmaceutical excipient to the human patient in an amount sufficient to reduce the human patient's coronary perfusion pressure wherein the coronary ischemia is accompanied by coronary microcirculatory vasoconstriction.

9. The method of claim 8 wherein the pharmaceutical composition is a liquid pharmaceutical composition.

10. The method of claim 9 wherein the liquid pharmaceutical composition is administered orally, by i.v. perfusion or by an iv bolus.

11. The method of claim 8 wherein a dose of the liquid pharmaceutical composition includes up to about 1 milligram/mL of (1-{9-[(2R,3R,4S,5R)-3,4-dihydroxy-5-(hydroxymethyl)oxolan-2-yl]-6-aminopurin-2-yl}pyrazol-4-yl)-N-methylcarboxamide.

12. The method of claim 8 wherein the pharmaceutical composition dose is in the form of at least one tablet that is administered orally.

13. The method of claim 12 wherein the at least one tablet includes from 10 mg to 2 g (1-{9-[(2R,3R,4S,5R)-3,4-dihydroxy-5-(hydroxymethyl)oxolan-2-yl]-6-aminopurin-2-yl}pyrazol-4-yl)-N-methylcarboxamide.

14. The method of claim 8 wherein the administration of the at least one dose decreases coronary perfusion pressure by at least 10%.
